(19) United States
(12) Patent Application Publication
(10) Pub. No.: US 2019/0159757 A1
(43) Pub. Date: May 30, 2019

(54) PROBE SLEEVE, PIPE SOCKET, PIPE SOCKET ASSEMBLY, AND METHOD FOR WRAPPING THE PROBE SLEEVE AROUND THE PIPE SOCKET

(71) Applicant: Qin WANG, Suzhou, Jiangsu (CN)

(72) Inventor: Qin WANG, Suzhou, Jiangsu (CN)

(21) Appl. No.: 16/320,715

(22) PCT Filed: Jul. 29, 2016
(86) PCT No.: PCT/CN2016/092350

§ 371 Date: Jan. 25, 2019

(51) Int. Cl. A61B 8/00; A61B 8/12

(52) U.S. Cl. CPC A61B 8/4422; A61B 2562/247; A61B 2562/12; A61B 8/12

(57) ABSTRACT

A probe sleeve (1), a pipe socket (2), a pipe socket assembly, and a method for wrapping the probe sleeve (1) around the pipe socket (2), wherein the probe sleeve (1) comprises a large diameter section (11) and a small diameter section (12); the free end of the large diameter section (11) is an open end (111), and the free end of the small diameter section (12) is a closed end (121). The probe sleeve (1), which is an intracavitary probe sleeve/transesophageal probe sleeve, can be used with the pipe socket (2) and as a surrogate of a condom, and can achieve an aseptic operation in an operative process. The method for wrapping the probe sleeve (1) around the pipe socket (2), which wraps the probe sleeve (1) around the pipe socket (2), is used for an ultrasonic B-ultrasound operation, and can meet the requirements of an aseptic operation.

### TECHNICAL FIELD

The present invention relates to the technical field of ultrasonic B-ultrasound operations, and specifically relates to a probe sleeve, a pipe socket, a pipe socket assembly, and a method for wrapping the probe sleeve around the pipe socket.

### BACKGROUND ART

At present, there is no intracavitary probe sleeve/transesophageal probe sleeve on the market: 1. in most cases where the probe enters the cavity (anus or vagina), a condom is used as the surrogate cover; 2. in cases where the probe enters the esophagus, the B-ultrasound probe enters the esophagus directly after disinfection, without using a condom or any probe protective sleeve. However, the use of a condom instead of the intracavitary probe sleeve has the following problem: during an operation that requires asepsis, the wrapping of the condom cannot be done aseptically. Specifically, in the wrapping process, the condom will touch the B-ultrasound probe and the doctor's gloves, so that these contact surfaces all become non-aseptic areas, thereby increasing the risk in the operation.

### SUMMARY OF THE INVENTION

Currently, there is no intracavitary probe sleeve/transesophageal probe sleeve, and a condom is used as the surrogate of the intracavitary probe sleeve, or the esophageal probe does not use any probe protective sleeve, so that an aseptic operation cannot be achieved. In order to overcome the above defects in the prior art, the present invention provides a probe sleeve, a pipe socket, a pipe socket assembly, and a method for wrapping the probe sleeve around the pipe socket, wherein, before an operation, the B-ultrasound probe is inserted into the probe sleeve from a pipe socket body, the probe sleeve automatically deploys from a mounting section on the pipe socket just by pushing the B-ultrasound probe straight forward with a little force, and the outer wall of the probe sleeve does not contact any portion, being in a truly pollution-free, aseptic state, so that an aseptic operation can be achieved in an operative process.

In order to achieve the above object, the present invention adopts the following technical solution:

a probe sleeve comprising a large diameter section and a small diameter section, wherein

the free end of the large diameter section is an open end, and the free end of the small diameter section is a closed end.

Preferably, the probe sleeve further comprises a tapered transition section connected between the large diameter section and the small diameter section.

The probe sleeve, which is an intracavitary probe sleeve/transesophageal probe sleeve, can replace a condom to achieve an aseptic operation in an operative process.

A pipe socket for use together with the probe sleeve according to any one of the above, with an upper portion being a pipe socket body and a lower portion being a mounting section, wherein

an upper end of the mounting section is provided with an annular protuberance.

Preferably, an upper portion of the mounting section is a cylindrical section, and a lower portion of the mounting section is a tapered section.

The pipe socket, which is used in cooperation with the above probe sleeve, can achieve an aseptic operation in an ultrasonic B-ultrasound operation.

A pipe socket assembly comprising the probe sleeve according to any one of the above and the pipe socket according to any one of the above, wherein

the large diameter section of the probe sleeve is sleeved on the mounting section, and

the small diameter section of the probe sleeve is wrapped around the mounting section.

Preferably, the pipe socket assembly further comprises a rubber ring, wherein

the rubber ring can be sleeved on the bottom of the pipe socket body and stuck above the annular protuberance, for fixing the probe sleeve to the pipe socket. The annular protuberance can prevent the rubber ring from slipping off the pipe socket.

A method for wrapping a probe sleeve around a pipe socket, comprising steps of:

A. inserting the mounting section of the pipe socket into the large diameter section of the probe sleeve;

B. fixing the open end of the large diameter section of the probe sleeve to the bottom of the pipe socket body at a position above the annular protuberance with the rubber ring;

C. pushing the probe sleeve up until the large diameter section of the probe sleeve is completely sleeved on the mounting section of the pipe socket; and

D. wrapping the small diameter section of the probe sleeve around the mounting section of the pipe socket.

The method for wrapping the probe sleeve around the pipe socket according to the present invention, which is used to wrap the probe sleeve around the pipe socket, is used for an ultrasonic B-ultrasound operation, and can meet the requirements of an aseptic operation.

The present invention has the following advantageous effects: the probe sleeve, which is an intracavitary probe sleeve/transesophageal probe sleeve, can be used with the pipe socket and as a surrogate condom, and can achieve an aseptic operation in an operative process. Likewise, the method for wrapping the probe sleeve around the pipe socket according to the present invention, which is used to wrap the probe sleeve around the pipe socket, is used for an ultrasonic B-ultrasound operation, and can meet the requirements of an aseptic operation.

### BRIEF DESCRIPTION OF THE DRAWINGS

FIG. 1 is a structural schematic diagram of a probe sleeve according to an embodiment of the present invention;

FIG. 2 is a structural schematic diagram of a pipe socket according to an embodiment of the present invention;

FIG. 3 to FIG. 4 are both structural schematic diagrams of a pipe socket assembly according to an embodiment of the present invention (the mounting states of the probe sleeve are different);

FIG. 5 is a schematic flow chart of a method for wrapping the probe sleeve around the pipe socket according to an embodiment of the present invention.

In the figures, 1—probe sleeve; 11—large diameter section; 111—open end; 12—small diameter section; 121—closed end; 13—transition section; 2—pipe socket; 21—pipe socket body; 22—mounting section; 221—cylindrical section; 222—tapered section; 23—annular protuberance; 3—rubber ring.

### DETAILED DESCRIPTION OF THE EMBODIMENTS

Hereinafter, the structures of the present invention will be explained in detail with reference to the figures.

As shown in FIG. 1, a probe sleeve 1 provided by an embodiment of the present invention comprises a large diameter section 11 and a small diameter section 12, which are an integral structure, wherein the free end of the large diameter section 11 is an open end 111, and the free end of the small diameter section 12 is a closed end 121.

The probe sleeve 1 provided by the embodiment of the present invention further comprises a tapered transition section 13 connected between the large diameter section 11 and the small diameter section 12, and when the probe sleeve 1 comprises the large diameter section 11, the tapered transition section 13 and the small diameter section 12, the large diameter section 11, the transition section 13 and the small diameter section 12 may be an integral structure.

The probe sleeve 1 provided by the embodiment of the present invention may be used as an intracavitary probe sleeve/transesophageal probe sleeve, and can replace a condom to achieve an aseptic operation in an operative process.

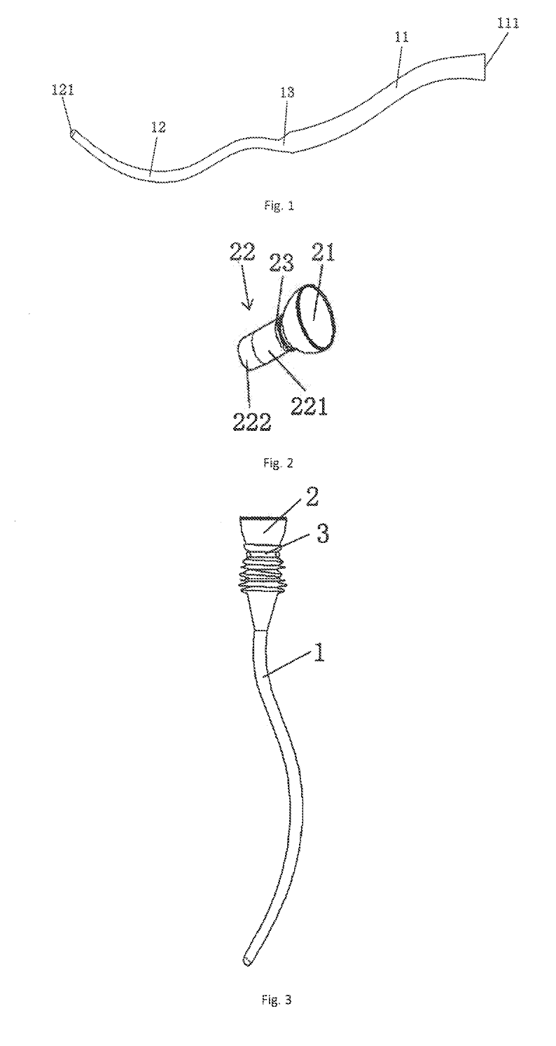

As shown in FIG. 2, a pipe socket 2 for use together with the above probe sleeve 1, as provided by an embodiment of the present invention, has an upper portion being a pipe socket body 21 and a lower portion being a mounting section 22, wherein an upper end of the mounting section 22 is provided with an annular protuberance 23. An upper portion of the mounting section 22 is a cylindrical section 221, and a lower portion of the mounting section 22 is a tapered section 222.

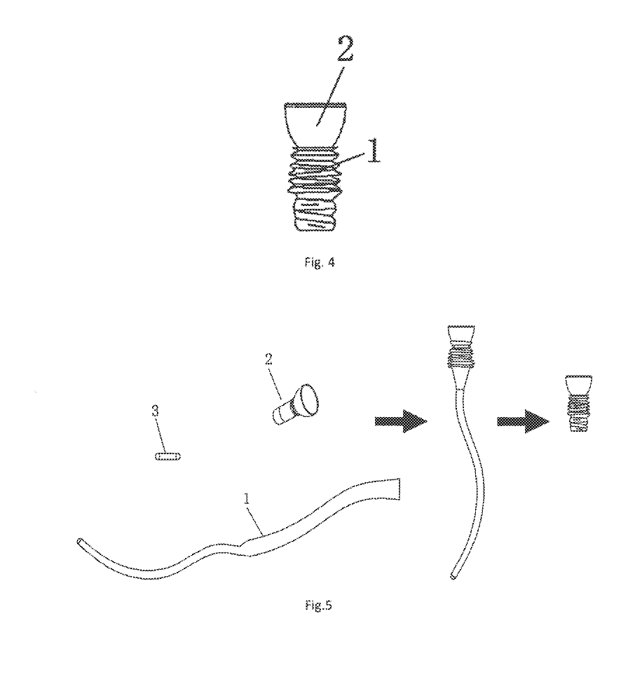

As shown in FIG. 3 to FIG. 4, a pipe socket assembly provided by an embodiment of the present invention comprises the above probe sleeve 1, the above pipe socket 2, and a rubber ring 3, wherein

the large diameter section 11 of the probe sleeve 1 is sleeved on the mounting section 22, wherein

the rubber ring 3 is sleeved on the bottom of the pipe socket body 21 and stuck above the annular protuberance 23, for fixing the probe sleeve 1 to the pipe socket 2, and the small diameter section 12 of the probe sleeve 1 is wrapped around the mounting section 22. In a specific operation, the large diameter section 11 of the probe sleeve 1 is preferably sleeved on the cylindrical section 221 of the pipe socket 2, and the small diameter section 12 of the probe sleeve 1 is preferably wrapped around the tapered section 222.

It should be noted that, since the upper end of the mounting section is provided with the annular protuberance 23, when the rubber ring 3 is sleeved on the bottom of the pipe socket body 21, the rubber ring 3 is stuck above the annular protuberance 23, so that the rubber ring 3 does not slide down.

As shown in FIG. 5, a method for wrapping the probe sleeve around the pipe socket, as provided by an embodiment of the present invention, comprises steps of:

A. inserting the mounting section 22 of the pipe socket 2 into the large diameter section 11 of the probe sleeve 1;

B. fixing the open end 111 of the large diameter section 11 of the probe sleeve 1 to the bottom of the pipe socket body 21 at a position above the annular protuberance 23 with the rubber ring 3;

C. pushing the probe sleeve 1 up until the large diameter section 11 of the probe sleeve 1 is completely sleeved on the mounting section 22 of the pipe socket 2; and

D. wrapping the small diameter section 12 of the probe sleeve 1 around the mounting section 22 of the pipe socket 2.

During an operation, there is only a need to insert the B-ultrasound probe from the pipe socket body 21, so that the probe sleeve 1 is automatically detached from the pipe socket 2 and completely covers the surface of the B-ultrasound probe, and the outer surface of the probe sleeve 1 does not contact any thing in the process of inserting the B-ultrasound probe, so as to achieve a truly aseptic state.

The above are only preferred embodiments of the present invention and are not intended to limit the present invention. Any modifications, equivalent substitutions, simple improvements and the like made in the essential contents of the present invention shall be included in the scope of protection of the present invention.

1. A probe sleeve, characterized in that, the probe sleeve (1) comprises a large diameter section (11) and a small diameter section (12), wherein
the free end of the large diameter section (1) is an open end (111), and the free end of the small diameter section (12) is a closed end (121).

2. The probe sleeve according to claim 1, characterized in that, the probe sleeve (1) further comprises a tapered transition section (13) connected between the large diameter section (11) and the small diameter section (12).

3. The probe sleeve of claim 1 further comprises a pipe socket, characterized in that, an upper portion of the pipe socket (2) is a pipe socket body (21), and a lower portion of the pipe socket (2) is a mounting section (22), wherein
an upper end of the mounting section (22) is provided with an annular protuberance (23).

4. The probe sleeve of claim 3 wherein the pipe socket is characterized in that, an upper portion of the mounting section (22) is a cylindrical section (221), and a lower portion of the mounting section (22) is a tapered section (222).

5. A pipe socket assembly, comprising:
a probe sleeve (1) having a large diameter section (11) and a small diameter section (12); and
a pipe socket (2) having an upper portion and a lower portion, the upper portion is a pipe socket body (21) and the lower portion is a mounting section (22), wherein
the large diameter section (11) of the probe sleeve (1) is sleeved on the mounting section (22), and the small diameter section (12) of the probe sleeve (1) is wrapped around the mounting section (22).

6. The pipe socket assembly according to claim 5, characterized in that, the pipe socket assembly further comprises a rubber ring (3), wherein
the rubber ring (3) can be sleeved on the bottom of the pipe socket body (21) and stuck above the annular protuberance (23), for fixing the probe sleeve (1) to the pipe socket (2).

7. A method for wrapping a probe sleeve around a pipe socket, characterized by, comprising steps of:
A. inserting the mounting section (22) of the pipe socket (2) into the large diameter section (11) of the probe sleeve (1);
B. fixing the open end (111) of the large diameter section (11) of the probe sleeve (1) to the bottom of the pipe socket body (21) at a position above the annular protuberance (23) with the rubber ring (3);
C. pushing the probe sleeve (1) up until the large diameter section (11) of the probe sleeve (1) is completely sleeved on the mounting section (22) of the pipe socket (2); and
D. wrapping the small diameter section (12) of the probe sleeve (1) around the mounting section (22) of the pipe socket (2).
